(19) United States
(12) Patent Application Publication
(10) Pub. No.: US 2010/0022652 A1
(43) Pub. Date: Jan. 28, 2010

(54) Antimicrobial and Anticoagulant Compositions and Methods

(75) Inventors: Dinesh Aggarwal, Franklin, MA (US); Chirag B. Shah, North Attleboro, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/489,669

(22) Filed: Jun. 23, 2009

Related U.S. Application Data
(60) Provisional application No. 61/129,905, filed on Jul. 28, 2008.

(51) Int. Cl. A61K 31/198; A61P 7/02; A61P 31/04

(52) U.S. Cl. 514/566

(57) ABSTRACT

A composition may include a combination of at least one chelating agent and at least one antioxidant agent, the combination may have a fractional inhibitory concentration coefficient of about 0.5 or less and/or an INR of > about 2.82.

### FIELD

The present invention is directed to compositions and methods that may exhibit one or more of antimicrobial and anticoagulant properties. The present invention is also directed to compositions and methods including at least one chelating agent and at least one antioxidant.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

A variety of antimicrobial compositions and methods have been suggested. However, such compositions and methods possess various deficiencies and shortcomings.

For example, a number of compositions have been proposed which include various agents. One such agent that has been proposed is a chelating agent. In particular, ethylene diamine tetraacetic acid (hereafter “EDTA”) has been identified as a chelating agent of interest.

However, a need exists in the art for compositions and methods which have increased effectiveness in reducing and/or preventing development of unwanted microbial organisms, and/or that have anticoagulant properties.

### DEFINITIONS

As used herein, unless otherwise indicated, the terms “microbial organism” or “microbial” will be used to refer to microscopic organisms of matter, including fungal, bacterial and/or viral organisms. Thus, the term “antimicrobial” as used herein refers to a composition or agent that kills or otherwise inhibits the growth of such fungal, bacterial and/or viral organisms.

As used herein, unless otherwise indicated, the term “anticoagulant” as used herein refers to a composition or agent that inhibits the clotting tendencies of blood.

As used herein, unless otherwise indicated, “minimum inhibitory concentration” (hereafter “MIC”) means the minimum concentration of a particular substance or agent necessary to inhibit the growth of a particular microbial organism. MIC may relate to inhibition of microorganisms including, but not limited to Pseudomonas aeruginosa (ATCC 27853) and/or Staphylococcus aureus (ATCC 25923) organisms.

As used herein, unless otherwise indicated, “fractional inhibitory concentration” (hereafter “FIC”) is an interaction coefficient indicating whether the combined inhibitory effect of a particular combination of agents is synergistic, additive or antagonistic. Thus, for example, FIC=A+B; wherein A=(MIC of combination X+Y)/(MIC of agent X alone), and B=(MIC of combination X+Y)/(MIC of agent Y alone). The FIC is generally considered to be synergistic when A+B≦0.5, partially synergistic when 0.5<A+B<1.0, additive when A+B=1 and antagonistic when 1<A+B≦4.

It is to be understood that reference herein to first, second, third and fourth components (etc.) does not limit the present invention to embodiments where each of these components or constituents are physically separable from one another. For example, a single physical element or constituent of the invention may perform the functions of more than one of the claimed first, second, third or fourth components or constituents. Conversely, a plurality of separate physical elements working together may perform the functions of one of the claimed first, second, third or fourth components or constituents. Similarly, reference to first, second (etc.) method steps does not limit the invention to only separate steps. According to the invention, a single method step may satisfy multiple steps described herein. Conversely, a plurality of method steps could, in combination, constitute a single method step recited herein. In addition, the steps of the method are not necessarily limited to the order in which they are described or claimed herein.

### SUMMARY

The present invention may optionally possess one or more of the following benefits or advantages: (i) compositions and methods which exhibit enhanced antimicrobial activity relative to compositions and methods involving EDTA alone; (ii) compositions and methods which are effective and safe for human administration, as well as being readily available; (iii) compositions and methods in which the active ingredients are water-soluble thereby facilitating formulation and manufacture; and/or (iv) compositions and methods that possess anticoagulant properties.

According to one aspect, the present invention is directed to a composition or structure which comprises, consists essentially of, or consists of an EDTA chelating agent and N-Acetyl Cysteine (hereafter “NAC”) antioxidant, and/or salts or derivatives thereof, and an optional carrier. According to yet another aspect, the present invention is directed to methods for using any of the above-mentioned compositions, including use of these compositions on or in medical devices, including use within a conduit to prevent thrombus formation. According to further aspects, the present invention is directed to formulations, articles and devices, such as medical devices, incorporating the compositions of the present invention.

More particularly, according to one optional aspect, the present invention provides a composition comprising a combination of at least one chelating agent comprising EDTA or a salt thereof, and at least one antioxidant agent comprising comprises N-acetyl Cysteine or a salt thereof.

According to another optional aspect, the present provides a method comprising formulating a composition comprising a combination of at least one chelating agent comprising EDTA or a salt thereof, and at least one antioxidant agent comprising N-acetyl Cysteine or a salt thereof, and applying the composition to a surface of an inanimate object, or topically applying the composition to a human.

### BRIEF DESCRIPTION OF THE DRAWINGS

FIG. 1 shows the percentage relationship of species of an EDTA salt in relation to the pH of a solution thereof.

FIG. 2 is a table reporting the results of antimicrobial synergy evaluations of compositions, including compositions according to the present invention, with respect to the Pseudomonas aeruginosa microorganism.

FIG. 3 is a table reporting the results of antimicrobial synergy evaluations of compositions, including compositions according to the present invention, of the present invention with respect to the Staphylococcus aureus microorganism.

FIG. 4 is a table reporting the results of potential antimicrobial activity via a time kill methodology of compositions, including compositions according to the present invention, with respect to the Pseudomonas aeruginosa microorganism.

FIG. 5 is a table reporting the results of potential antimicrobial activity via time kill methodology of compositions, including compositions according to the present invention, with respect to the Staphylococcus aureus microorganism.

FIG. 6 is a table reporting the results of anticoagulant activity of DEDTA and NAC, individually, and of compositions, including compositions according to the present invention, including both DEDTA and NAC, via a prothrombin assay.

FIG. 7 is a graphical representation of the INR values for DEDTA and an NAC, individually, when compared with controls via a prothrombin assay.

FIG. 8 is a graphical representation of the effect of 2 wt. % NAC on the anticoagulant activity of DEDTA, NAC and DEDTA+NAC based formulations.

FIG. 9 is a graphical representation of the effect of 4 wt. % NAC on the anticoagulant activity of DEDTA, NAC and DEDTA+NAC based formulations.

### DETAILED DESCRIPTION

In a broader sense, the present invention is directed to compositions which include at least one chelating agent comprising EDTA or a salt thereof, and at least one antioxidant comprising NAC.

By way of non-limiting example, “EDTA” includes variations of EDTA such as, for example, disodium EDTA (“DEDTA”), combinations thereof and the like, are contemplated. Soluble salts of EDTA may be used in compositions of the present disclosure. Sodium salts of EDTA are commonly available, including di-sodium, and tri-sodium salts, although other EDTA salts, including ammonium, di-ammonium, potassium, di-potassium, cupric di-sodium, magnesium di-sodium, ferric sodium, and combinations thereof, may be used, provided they have the desired antimicrobial properties. Various combinations of EDTA salts may also be used, and may be preferred for particular applications.

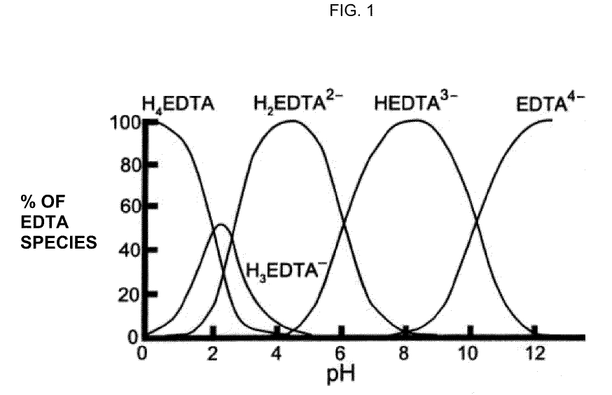

At physiological pH, the sodium salts of EDTA exist as a combination of di-sodium and tri-sodium EDTA, with the tri-sodium salt(s) of EDTA being predominant. In the U.S., pharmaceutical “di-sodium” EDTA prepared for injection has generally been titrated with sodium hydroxide to a pH of 6.5 to 7.5. At this pH, the EDTA solution actually comprises primarily tri-sodium EDTA, with a lesser proportion of the di-sodium salt. FIG. 1 shows the percentage relationship of species of EDTA sodium salts in relation to the pH of the solution. Other compositions comprising sodium salts of EDTA that are used in medical or healthcare applications are generally adjusted to a pH that is substantially physiological.

EDTA is used at low concentrations as a stabilizer or preservative in many compositions. Compositions of the present invention comprise generally higher concentrations of EDTA. Compositions of the present invention may include any suitable level of chelating agent or EDTA. Accordingly, by non-limiting example, compositions of the present invention may comprise, in weight percent, less than or equal to about 4.0%. According to further optional embodiments, the amount of EDTA, or a salt thereof, may be about 4% by weight, about 2% by weight, about 0.004%-0.08% by weight, or about 0.004%-0.0125% by weight.

The NAC antioxidant can be a derivative of the naturally occurring amino acid N-cysteine. NAC is a sulfhydryl group donor and is therefore considered an antioxidant. A number of salts and derivatives of NAC are also contemplated. In particular, salts of NAC with inorganic cations such as sodium, alkyl ammonium cations, and other pharmaceutically acceptable cations may be utilized. Possible derivatives of NAC include its esters, thioesters and thioethers. In some embodiments, both the thiol group and the carboxylate group of the NAC may be derivatized. In yet other active derivatives, the acetyl group of NAC may be replaced by another acyl group.

NAC may be included in any suitable amount. By way of non-limiting example, compositions of the present invention may include about, in weight percent, less than or equal to about 4.0% by weight. According to further embodiments, the amount of NAC, or a salt thereof, may be about 0.031%-about 4.0% by weight, about 2.0%-about 4.0% by weight, about 0.125%-about 2.0% by weight, or about 0.125%-about 1.0% by weight.

The above-described compositions of the present invention may be combined with a suitable carrier. Suitable carriers include, but are not limited to, water, solvents, gels, creams, hydrogels, foams, plastics, metals, ceramics, polymers, fibrous materials, woven materials, and nonwoven materials.

Compositions formed according to the principles of the present invention also possess anticoagulant properties. Thus, by way of non-limiting example, compositions of the present invention may be utilized in the form of a catheter lock solution. Typically, the EDTA and NAC components are dissolved in water as a carrier, although other carriers may be used. Substances such as thrombolytics, sodium, alcohol, or reagents may also be added to the deionized water/EDTA and NAC solution.

The compositions of the present disclosure may also be provided in a substantially “dry” form, such as a substantially dry coating on a surface of tubing, or a conduit, or a medical device such as a catheter or conduit, or a container, or the like.

The compositions of the present invention may also be incorporated into a wound dressing. A wound dressing formed according to the principles of the present invention can be generally formed from one or more discrete layers. Each of the one or more layers can be formed from any suitable material and/or construction. For example, the one or more layers can be formed from a fibrous, film-like, or foam material. With respect to fibrous materials, they can be woven or nonwoven materials. The fibers can be selected from natural fibers, synthetic fibers, and combinations of the two. By way of non-limiting example, suitable materials which can be utilized to form the one or more layers of the present invention include: cellulose, alginates, cotton, Rayon, Nylon, acrylic, polyester, polyurethane, polyurethane foam, and combinations thereof. Wound dressings can, of course, include additional active ingredients or agents such as, for example, a therapeutic agent, an organoleptic agent, a growth factor, an analgesic, a tissue scaffolding agent, a haemostatic agent, a protein inhibitor, collagen, enzymes, an anti-thrombogenic agent, an anesthetic, an anti-inflammatory agent, an anticancer agent, a vasodilation substance, a wound healing agent, an angiogenic agent, an angiostatic agent, an immune boosting agent, a skin sealing agent, an agent to impart bactericidal or bacteriostatic activity, an electron transfer agent to destabilize or destroy the metabolic action of microbes and/or biofilm formation, combinations thereof and the like. Release of active agents may be triggered by a variety of means, such as, for example, an electric field or signal, temperature, time, pressure, moisture, light (e.g., ultra-violet light), ultrasound energy, sonication, combinations thereof and the like.

According to the present invention, the above-mentioned compositions may be combined directly with the material forming the one or more layers of the wound dressing. Alternatively, any of the above-mentioned agents may be contained, and subsequently released, by a delivery agent. Any suitable delivery agent can be utilized. By way of non-limiting example, suitable delivery agents include: a hydrogel, phosphate glass, powdered starch, or a starch film.

The present invention is also directed to methods of utilizing the above-described compositions for active abatement, or prophylaxis, of undesirable microbial organisms. Thus, by way of non-limiting example, the compositions of the present invention may be applied to a surface of an inanimate object, such as a piece of medical equipment or a medical device (e.g. catheter), a piece of industrial equipment, or household objects. Alternatively, or in addition, compositions of the present invention may be topically applied to human subject.

Methods for inhibiting the growth and proliferation of microbial organisms include inhibiting the formation and proliferation of biofilms.

Certain aspects of the present invention will now be further described by reference to the following non-limiting examples. The following examples are for purpose of illustration only.

### EXAMPLES

Experiments were conducted showing an unexpected antimicrobial synergism of compositions comprising EDTA and NAC formulated according to the present invention.

A first set of experiments were conducted involving screening experiments using checkerboard titration to assess if certain combinations of EDTA and NAC possess a FIC index value of ≦1. The combinations having an FIC index of ≦0.5 are considered synergistic while a FIC index of >0.5 but ≦1 are considered partially synergistic. See, e.g., Ann Clin Micorbiol Antimicrob, 2006, 5: p. 25 & J Infect Chemother, 2006, 12 (4); P. 172-176. The method used was similar to a NCCLS micro-dilution procedure (See, e.g., CLSI, Methods for Dilution Antimicrobial Susceptibility Tests for Bacteria that Grow Aerobically; Approved Standard Clinical Laboratory Standard Institute (CLSI): 6(2).

First, potential antimicrobial synergy in a composition having a combination of EDTA and NAC components was investigated with respect to the Pseudomonas aeruginosa (ATCC 27853) microorganism.

The following materials are utilized in the investigation are listed in Table I below.

TABLE I

| Reagents/chemicals | Manufacturer | Catalog# |
|---|---|---|
| disodium EDTA | J. T. Baker | 8994-01 |
| (DEDTA) | | |
| N-Acetyl Cysteine | Acros | 16028-0500 |
| (NAC) | | |
| Muller-Hinton Broth | Difco | 275730 |
| (MHB) | | |

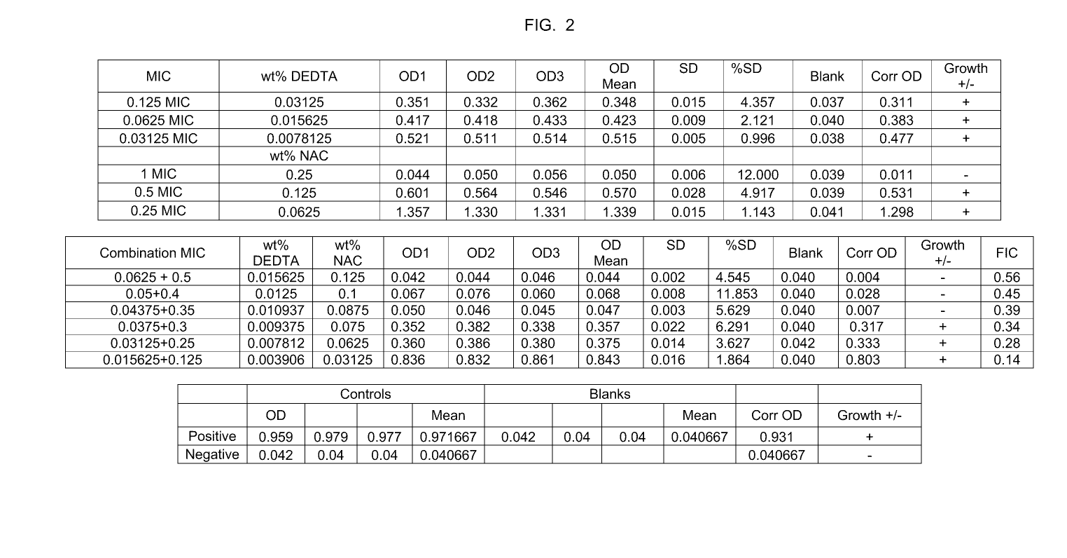

Stock solutions were prepared by a method similar to that described Ann Clin Micorbiol Antimicrob, 2006, 5: p. 25 & J Infect Chemother, 2006, 12 (4); P. 172-176. According to this method the stock solutions are prepared having a concentration equivalent or higher than twice the MIC of the components among which synergy is to be tested. The MIC of DEDTA and NAC against Pseudomonas aeruginosa (ATCC 27853) microorganism is 0.25 wt % and 0.25 wt %, respectively. For the assay, DEDTA- and NAC-containing solutions were prepared at various concentrations, as indicated in the second and third columns of the middle portion of the table of FIG. 2.

The results and observations of this investigation are reported in FIG. 2. Those compositions having a FIC less than 1.0 are considered at least partially synergistic with respect to antimicrobial activity, and those compositions with a FIC less than or equal to 0.5 are considered synergistic with respect to antimicrobial activity against Pseudomonas aeruginosa, and any composition reported herein exhibiting a partially synergistic or a synergistic FIC are considered as formulations within the scope of the present invention.

As seen from FIG. 2, the antimicrobial activity with respect to Pseudomonas aeruginosa of certain compositions containing both EDTA and NAC components was at least partially synergistic as indicated by the reported FIC values.

Second, antimicrobial synergy in compositions according to the present invention having a combination of EDTA and NAC components was investigated with respect to the Staphylococcus aureus (ATCC 25923) organism.

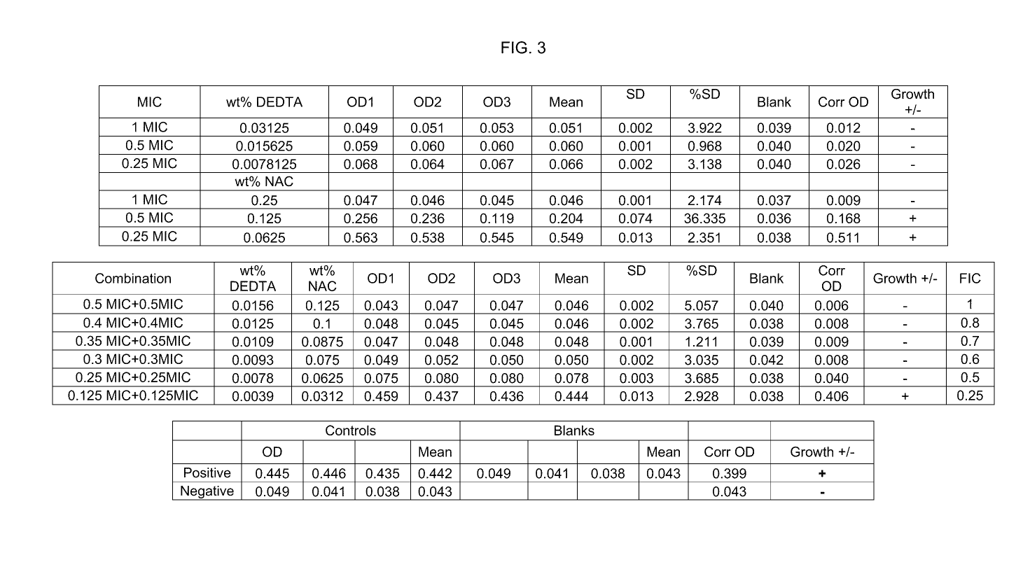

The same materials described above in connection with the first example were utilized. The same methodology described above in connection with the first example was utilized. Final solutions were prepared having at various concentrations, as indicated in the second and third columns of the middle portion of the table of FIG. 3.

The results and observations of this investigation are reported in FIG. 3.

As indicated therein, the antimicrobial activity with respect to Staphylococcus aureus of certain composition containing both EDTA and NAC components was synergistic, or at least partially synergistic (a FIC less than 1.0 are considered at least partially synergistic, and those compositions with a FIC less than or equal to 0.5 are considered synergistic). Any composition reported herein exhibiting a partially synergistic or a synergistic FIC are considered as formulations within the scope of the present invention

The “OD” values contained in FIGS. 2 and 3 refer to unitless optical density values. These values are representative of a measure of an amount of light at a certain wavelength that can be transmitted through a sample or solution. The optical density measurements reported in FIGS. 2-3 were measured using light with a wavelength of approximately 630 nm. As the solutions get turbid, a higher optical density values generated. Generally speaking, increased turbidity, and thus higher optical density readings, are indicative of the presence of micro organisms in the solution. During analysis of the samples, optical density measurements above a predetermined cutoff value is indicative that there are microorganisms present in the solutions or samples, and are thus characterized by positive growth (+), while optical density readings which fall below the cutoff value can be characterized by negative growth (−). A number of the OD measurements were made (OD1, OD2, OD3), and the mean value calculated.

Antimicrobial synergy/partial synergy in the composition having EDTA and NAC components was further investigated via a time kill method (TM-4339-082) utilizing Pseudomonas aeruginosa (ATCC 27853) microorganism. According to this method the test material or a dilution of the test material is brought into contact with the known population of microorganisms for a specified period of time at a specified temperature. Following exposure, the surviving microorganisms are enumerated. The log 10 reduction or recovery, from either an initial microbial population, or test blank, is calculated according to the standard set forth in ASTM, Standard “Guide for Antimicrobial Activity Using a Time-Kill Procedure,” 2003,E2315-03: p. 1-5.

Materials stated in Table 1 were utilized in this investigation.

8 wt % DEDTA and 8 wt % NAC stock solutions were prepared. These solutions were further diluted to the concentrations stated in the Table 2 below:

| Final Solution | Conc. wt % | pH |
|---|---|---|
| 0.5 MIC - NAC | 0.125 wt % | 2.58 |
| 0.25 MIC - NAC | 0.0625 wt % | 2.70 |
| 0.5 MIC - DEDTA | 0.125 wt % | 4.81 |
| 0.25 MIC - DEDTA | 0.0625 wt % | 4.86 |

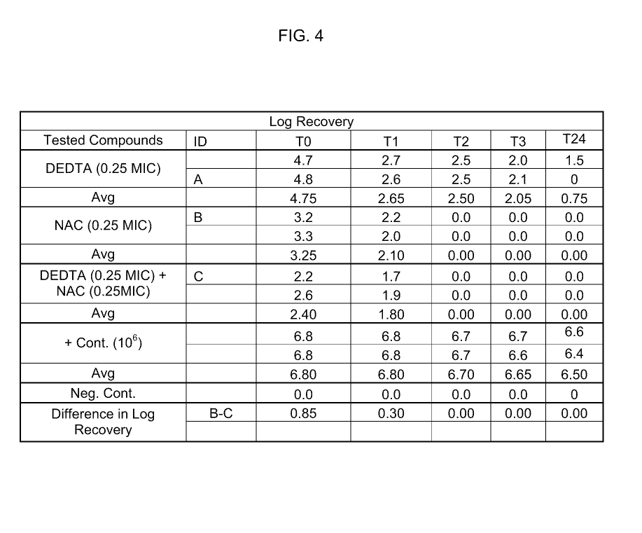

The results and observations of this investigation are reported in FIG. 4.

A rate of kill assay can determine whether combinations are synergistic or not. In these assays the formulations are first exposed to organisms for a desired time (the current formulations readings were taken at 0, 1, 2, 3 and 24 hrs). Then a sample of the organisms and formulation mixture is serially diluted and plated to assess the log recovery (a measurement of the level of organism growth). The organisms are allowed to grow and are checked for growth/log recovery after 24 hrs. The log recovery values obtained for individual components were compared with the combinations. Any combinations having ≧2 reduction in log recovery value when compared with the most active compound used in the combination at any time point tested were labeled as synergistic (see, e.g., International Journal of Antimicrobial Agents, 15 (2000) 125-129; and BMC Infect Dis. 2007: 7: p. 111; and Antimicrob Agents Chemother, 2005, 49(7): p. 2959-64.

As evident from the data therein, the antimicrobial activity with respect to Pseudomonas aeruginosa of the composition containing both EDTA and NAC components was slightly more effective when compared with the most active compound i.e., NAC. The NAC (0.25 MIC) by itself resulted in log recovery of 2.10 at time T1 while the DEDTA (0.25 MIC)+NAC (0.25 MIC) resulted in log recovery of 1.80 thus indicating higher kill for the combination formulation.

Antimicrobial synergy/partial synergy in compositions having EDTA and NAC components was further investigated according to the same methodology as the previous example, utilizing Staphylococcus aureus (ATCC 25923) organism.

Materials stated in table 1 were utilized.

8 wt % DEDTA and 8 wt % NAC stock solutions were prepared. These solutions were further diluted to concentrations stated in Table 3 below:

| Final Solution | Conc. wt % | pH |
|---|---|---|
| 0.5 MIC - NAC | 0.125 wt % | 2.58 |
| 0.25 MIC - NAC | 0.0625 wt % | 2.70 |
| 0.125 MIC - NAC | 0.0312 wt % | 2.83 |
| 0.0625 MIC - NAC | 0.0156 wt % | 2.94 |
| 0.5 MIC - DEDTA | 0.0156 wt % | 4.73 |
| 0.25 MIC - DEDTA | 0.0078 wt % | 4.89 |
| 0.125 MIC - DEDTA | 0.0039 wt % | 4.96 |

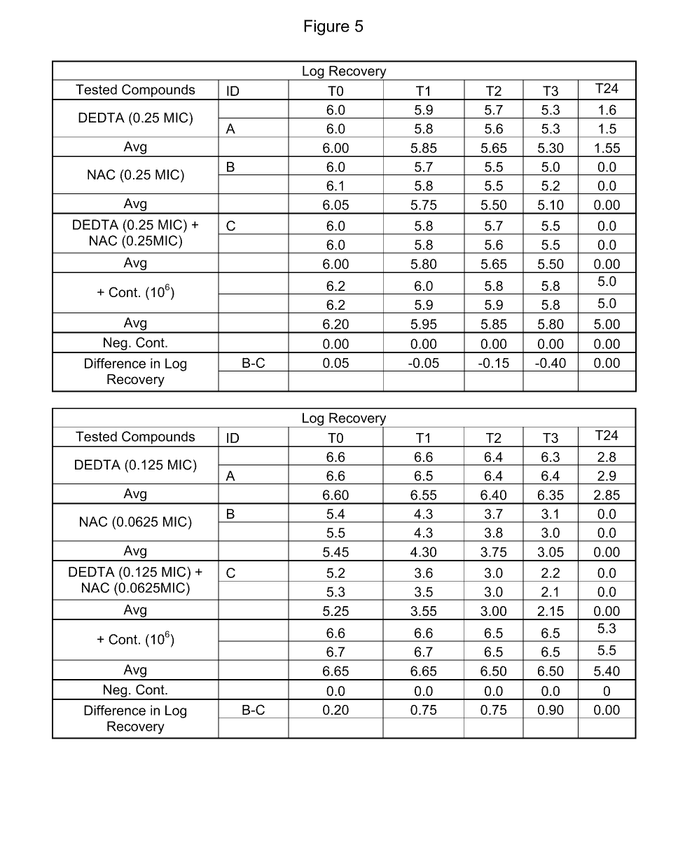

The results and observations of this investigation are reported in FIG. 5.

As indicated therein, the antimicrobial activity with respect to Staphylococcus aureus of the composition containing both EDTA and NAC components was more effective when compared with the most active compound i.e. NAC. The NAC (0.0625 MIC) by itself resulted in log recovery of 3.05 at time T3 while the DEDTA (0.125 MIC)+NAC (0.0625 MIC) resulted in 2.15 log recovery thus indicating a higher kill for the combination formulation.

Potential anticoagulant or anticoagulant synergy in compositions having a combination of EDTA and NAC components was investigated via a prothrombin time (PT) assay. The methodology utilized is described below.

8 wt % DEDTA and 8 wt % NAC solutions were prepared in DI water utilizing the materials set forth in Table 1. These solutions were then diluted/mixed to obtain the following solutions:

| | | Concentration |
|---|---|---|
| No. | Composition | (wt %) |
| 1 | DEDTA | 1.0 |
| 2 | DEDTA | 2.0 |
| 3 | DEDTA | 4.0 |
| 4 | DEDTA | 8.0 |
| 5 | NAC | 2.0 |
| 6 | NAC | 4.0 |
| 7 | NAC | 8.0 |
| 8 | NAC + DEDTA | 2.0 + 2.0 |
| 9 | NAC + DEDTA | 2.0 + 4.0 |
| 10 | NAC + DEDTA | 4.0 + 2.0 |
| 11 | NAC + DEDTA | 4.0 + 4.0 |

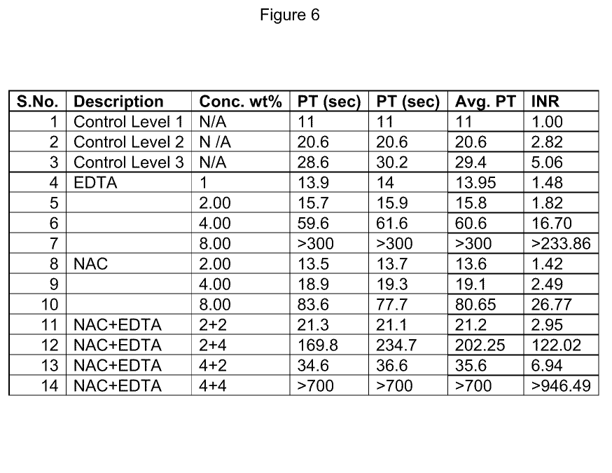

The results and observations of this investigation are reported in FIG. 6-9

The Prothrombin Time (PT) Assay primarily measures the time required for the clot formation in a test sample. Per the assay, the test samples first mixed with normal anticoagulated plasma in a ratio of 1:9 and incubated for 2 minutes at Room temperature. Then tissue thromboplastin with calcium ions is added to the test samples. The samples are then incubated for 2 minutes at 37° C. Tissue thromboplastin and calcium ions is added to normal anticoagulated plasma mixed with formulation, to initiate the clotting mechanism leading to formation of a fibrin clot. The coagulation analyzer is utilized to record the PT required for clot formation. The PT so obtained is then converted to International Normalized Ratio (INR) by utilizing the following formula:

INR=(PT_{Test Sample}/PT_{Control Level 1})^{ISI};

wherein ISI (International Sensitivity Index)—indicates the sensitivity of individual thromboplastin. The value of ISI utilized in the anticoagulant investigations reported herein is 1.65.

The PT required to form fibrin clot in normal plasma is between 10-13 seconds which translated to INR equal 1. If the deficiency exists within coagulation pathway (for example test blood contains heparin or other anticoagulant) the time required for clot formation will be prolonged. Any INR values ≧ that of abnormal plasma level 2 (e.g., TriniCHECK™ Level 2; Trinity Biotech) are considered indicative of deficiency within the coagulation pathway, and thus indicative of highly anticoagulative properties. Any INR values higher than 5.5 are considered indicative of highly anticoagulant behavior. Therefore, the strength of an anticoagulant can be characterized by the INR value, i.e., the larger the INR value the stronger the anticoagulant properties.

The concentrations given in the tables of FIG. 6 are the final concentrations of the reagents. Control level 1 (e.g., TriniCHECK™ Level 1; Trinity Biotech) is a lyophilized human plasma with characteristics similar to those of fresh normal plasma to be used as a normal control in the Prothrombin Time (PT), assay procedures. Control levels 2 and 3 (e.g. TriniCHECK Level 2™ and TriniCHECK Level 3™) are lyophilized human plasmas in which Factors II, VII, IX, and X have been selectively and partially removed, to be used as abnormal controls in the Prothrombin Time (PT) test (any INR values≧5.5 indicates very high anticoagulant capacity and is of very little or no clinical significance (Interact CardioVasc Thorac Surg 2007;6:390-396)).

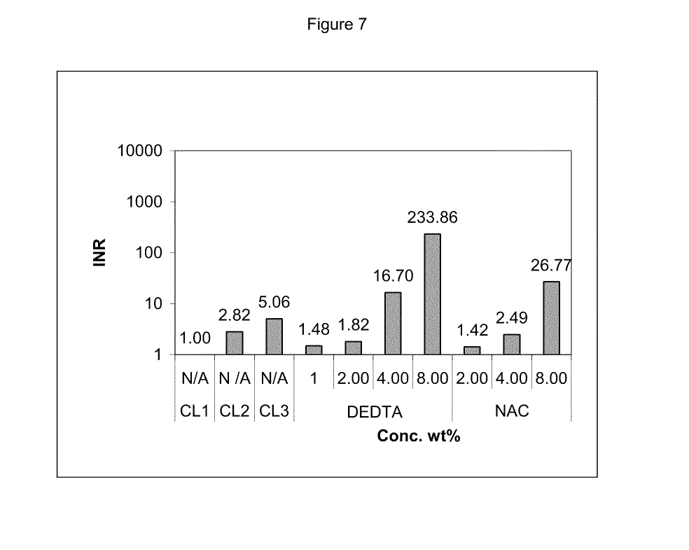

As indicated in FIG. 7, it is evident that (within the tested range):
INR for DEDTA increases with increase in its concentration (at 8 wt % the INR is >233);
INR for NAC increases with increase in its concentration (at 8 wt % the INR is >26);
In FIG. 7, CL1, CL2 and CL3 stand for Control Levels 1-3, respectively.

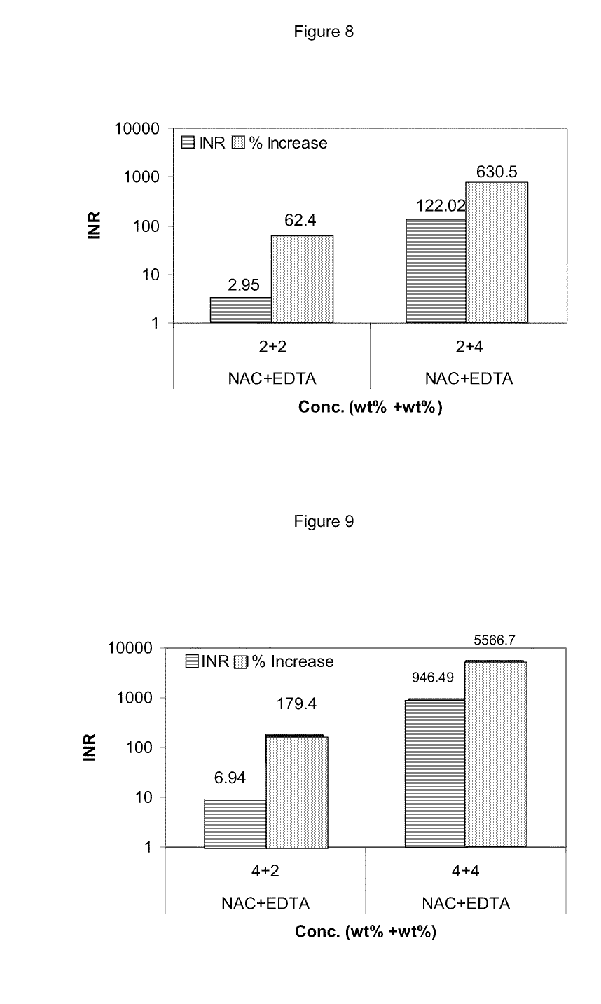

As indicated in FIGS. 8-9, and in comparison with FIG. 7, the anticoagulant activity of DEDTA was significantly enhanced by the addition of NAC. For example, when 2 wt % NAC was added to 4 wt % DEDTA, the formulation resulted in an INR value of about 122. This is 630% increase when compared with the most active compound in formulations with 4 wt % DEDTA alone (INR=16.7; FIG. 7). The effect is amplified when 4 wt % NAC is utilized instead of 2 wt %. As can be observed from FIG. 9, there is >5500% increase in INR when compared with the most active single compound in the formulation.

All numbers expressing quantities of ingredients, constituents, reaction conditions, and so forth used in the specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors as evident from the standard deviation found in their respective measurement techniques. None of the elements recited in the appended claims should be interpreted as invoking 35 U.S.C. §112, ¶6, unless the term “means” is explicitly used.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

We claim:

1. A composition comprising a combination of:
at least one chelating agent comprising EDTA or a salt thereof; and
at least one antioxidant agent comprising comprises N-acetyl Cysteine or a salt thereof.

2. The composition of claim 1, wherein the combination has a fractional inhibitory concentration coefficient of about less than 1.0.

3. The composition of claim 2, wherein the fractional inhibitory concentration coefficient is about 0.7 or less.

4. The composition of claim 3, wherein the fractional inhibitory concentration coefficient is about 0.6 or less.

5. The composition of claim 4, wherein the fractional inhibitory concentration is about 0.5.

6. The composition of claim 1, wherein the chelating agent comprises DEDTA.

7. The composition of claim 1, wherein the composition has an acidic pH.

8. The composition of claim 1, wherein the composition comprises, in weight percent, less than or equal to about 4.0% EDTA, or a salt thereof, and less than or equal to about 4.0% N-acetyl Cysteine, or a salt thereof.

9. The composition of claim 8, wherein the composition comprises more N-acetyl Cysteine, or a salt thereof, than EDTA, or a salt thereof.

10. The composition of claim 9, wherein the composition comprises about 2.0% EDTA, or a salt thereof, and about 4.0% N-acetyl Cysteine, or a salt thereof.

11. The composition of claim 8, wherein the composition comprises about 0.004%-0.08% by weight of EDTA, or a salt thereof, and about 0.031 %-0.125% by weight N-acetyl Cysteine, or a salt thereof.

12. The composition of claim 11, wherein the composition comprises about 0.004%-.0125% by weight of EDTA, or a salt thereof.

13. The composition of claim 1, further comprising an International Normalized Ratio (INR) calculated according to the following formula:
INR=(PT_{Test Sample}/PT_{Control Level 1})^{ISI};
wherein PT=the Prothrombin Time assay value; and ISI=International Sensitivity Index=1.65; and
INR>about 2.82.

14. The composition according to claim 13, wherein INR≧about 2.95.

15. The composition according to claim 14, wherein the INR≧about 5.5.

16. A method comprising:
formulating a composition comprising a combination of at least one chelating agent comprising EDTA or a salt thereof, and at least one antioxidant agent comprising N-acetyl Cysteine or a salt thereof; and
applying the composition to a surface of an inanimate object, or topically applying the composition to a human.

17. The method of claim 16, wherein the combination has a fractional inhibitory concentration coefficient of about less than 1.0.

18. The method of claim 17, wherein the fractional inhibitory concentration coefficient is about 0.7 or less.

19. The method of claim 18, wherein the fractional inhibitory concentration coefficient is about 0.6 or less.

20. The method of claim 19, wherein the fractional inhibitory concentration is about 0.5.

21. The method of claim 16, further comprising providing the composition with an acidic pH.

22. The method of claim 16, wherein the composition is formulated so as to comprise, in weight percent, less than or equal to about 4.0% EDTA, or a salt thereof, and less than or equal to about 4.0% N-acetyl Cysteine, or a salt thereof.

23. The method of claim 22, wherein the composition is formulated such that it comprises more N-acetyl Cysteine, or a salt thereof, than EDTA, or a salt thereof.

24. The method of claim 23, wherein the composition is formulated such that it comprise about 2.0% EDTA, or a salt thereof, and about 4.0% N-acetyl Cysteine, or a salt thereof.

25. The method of claim 16, wherein the composition is formulated such that it comprise about 0.004%-0.08% by weight of EDTA, or a salt thereof, and about 0.031 %-0.125% by weight N-acetyl Cysteine, or a salt thereof.

26. The method of claim 25, wherein the composition is formulated such that it comprise about 0.004%-0.0125% by weight of EDTA, or a salt thereof.

27. A method of preventing or impeding thrombus formation within a conduit, the method comprising: at least partially filling the conduit with a solution comprising a carrier and the composition of claim 1.

28. The method of claim 27, wherein the conduit comprises a catheter.

29. A composition comprising a combination of at least one chelating agent and at least one antioxidant agent, the combination having an International Normalized Ratio (INR) calculated according to the following formula:
INR=(PT_{Test Sample}/PT_{Control Level 1})^{ISI};
wherein PT=the Prothrombin Time assay value; and ISI=International Sensitivity Index=1.65; and
INR>about 2.82.

30. The composition according to claim 29, wherein INR≧about 2.95.

31. The composition according to claim 30, wherein the INR≧about 5.5.

32. In combination, the composition of claim 1, and a carrier.

33. The combination of claim 32, wherein the carrier comprises: water, solvents, gels, creams, hydrogels, foams, plastics, metals, ceramics, polymers, fibrous materials, woven materials, or nonwoven materials.
